(19) United States
(12) Patent Application Publication
(10) Pub. No.: US 2018/0133243 A1
(43) Pub. Date: May 17, 2018

(54) METHOD AND COMPOSITION FOR CRUDE FORMULATIONS OF FORTIFIED SUGAR FOR GLYCEMIC CONTROL

(71) Applicant: HOLISTA COLLTECH LTD, Perth, WA (US)

(72) Inventors: Daryl Thompson, Winter Haven, FL (US); Rajendran Manicka, Perth (AU)

(21) Appl. No.: 15/808,117

(22) Filed: Nov. 9, 2017

Related U.S. Application Data
(60) Provisional application No. 62/422,906, filed on Nov. 16, 2016.

(51) Int. Cl. A61K 31/733; A61K 31/7048; A61K 31/353; A61K 31/192; A61K 31/717; A23L 33/125

(52) U.S. Cl. CPC A61K 31/733; A61K 31/7048; A61K 31/353; A23V 2002/00; A61K 31/717; A23L 33/125; A61K 31/192

(57) ABSTRACT

A composition for glycemic control includes a sweetening agent, a polyphenol, and a starch. Examples of two formulations include but are not limited to i) a sweetening agent, gallic acid as the polyphenol and arabinoxylan as the starch; and ii) a sweetening agent, hesperitin or hesperidin as the polyphenol and inulin as the starch. Advantageously, the formulations include a combination of components to render an effective formulation that mitigates or rectifies hyperglycemic effects of high glycemic loads or intake.

### FIELD OF INVENTION

The present invention relates to a method and a composition for improved glycemic control and in particular a method and composition which has improved formulations of fortified sugar for glycemic control.

### BACKGROUND OF THE INVENTION

Diabetes is a major global contributor of death accounting for 1.5 million deaths annually with an addition 2.2 million attributed to high blood glucose levels. Diabetes has consistently risen globally in 1980 from 108 million to 422 million in 2015. The direct cause of developing diabetes/metabolic syndrome is the hyperglycemia or the direct negative effects of consuming large amounts of sugar. This quick rush of sugar triggers pro-inflammatory immune responses chronically lead to developing metabolic syndrome, obesity, diabetes and neurological disease (e.g. Type III diabetes).

### SUMMARY OF THE INVENTION

The present invention is directed to methods, formulations and uses of improved formulations of fortified sugar for glycemic control. The formulations include a combination of components to render an effective formulation that mitigates or rectifies hyperglycemic effects of high glycemic loads or intake. In one advantageous general form, the formulation has three distinct components: a sweetening agent, a polyphenol, and a starch. Examples of two formulations are:

i) a sweetening agent, gallic acid as the polyphenol and arabinoxylan as the starch; and

ii) a sweetening agent, hesperitin or hesperidin as the polyphenol and inulin as the starch.

The formulation may be combined with other ingredients to be incorporated into food stuff such as a beverage, batter, and a medical formulation just to name a few. A non-exclusive list of possible phenolic/polyphenol compounds include but are not limited to gallic acid, myricetin and hesperitin or hesperidin.

The present method and composition includes a specialized formulation of consumable sugar with additional biological buffering strategies to derive a carbohydrate based sweetening agent that reduces the glycemic impact of the carbohydrate base so that hyperglycemic effects in the blood stream are mitigated or significantly reduced. The formulation has three major class components; a carbohydrate base, a polyphenol, and a polysaccharide starch.

The purpose of the carbohydrate base is itself evident as it is the sweetening aspect of the formulation providing for its traditional use as a sweetening agent. The polyphenol aspect of the present formulation acts to inhibit the pro-inflammatory response via direct inhibition of the tnf-α pro-inflammatory pathway. The polyphenol has additional action in that it down-regulates the chronic effects of hyperglycemia in that it directs carbohydrate management for peak efficiency but reducing carbohydrates stored as fat and directing carbohydrates for immediate utilization of muscle tissue. The polysaccharide portion of the formulation further acts to control glycemic impact my acting as a substrate to carbohydrate converting enzymes such as alpha amylase and glucosidase.

The combination of three components (a sweetening agent, polyphenol, and a starch) when in an advantageous form results in mitigating the effects of hyperglycemia when the formulation is administered to an individual such as a human or animal suffering from hyperglycemia. In a specific advantageous form, the formulation mitigates against hyperglycemia such as metabolic syndrome, diabetes and neurological diseases.

The present formulation can be combined in a dry state suitable for addition into a food stuff during its manufacture. Examples of this include but are not limited to beverages, breads, candies, confections, coatings, medical foods and medicinal formulations.

The present formulation can be produced by heating a suitable sweetening agent, to form a liquefied state of the sweetener that is then added to the polyphenol and starch resulting in the components being embedded into the re-crystalized form of the sweetening agent. Examples of this include but are not limited to are table sugar, beverages, breads, candies, confections, coatings, medical foods and medicinal formulations.

In another aspect of the present formulation, the formulation is dispersed and carried in a liquid phase of the sweetening agent such as corn syrup. Examples of this include but are not limited to are beverages, breads, candies, confections, coatings, medical foods and medicinal formulations.

It is yet another aspect of the present formulation, in one advantageous form, that the sweetening agent be a carbohydrate base both a natural and semi synthetic nature. Examples of this include but are not limited to monosaccharides, disaccharides, oligosaccharides and artificial sweeteners. Examples include: fructose, galactose, sucrose, deoxyribose, diose, triose, tetrose, pentose, hexose, pentose, aldose, furanose, pyranose, glucofuranose, glucopyranose, sucrose, lactulose, lactose, maltose, cellobiose, chitobiose, kojibiose, nigerlose, isomaltose, sophorose, laminiaribiose, gentiobiose, turanose, maltulose, palatinose, mannobiose, melibiose, melibiulose, runtinose, xylobiose, fructo-oligosaccharide, galacto-oligosaccharide, manno-oligosaccharide, aspartame, cyclamate, saccharin, stevia, sucralose, acesulfame potassium, lead acetate, mogrosides, brazzein, curculin, erythritol, glycyrrhizin, glycerol, inulin, isomalt, lactitol, mabinlin, maltitol, miraculin, monatin, monellin, osladin, pentadin, sorbitol, tagetose, thaumatin, xylitol, advantame, alitame, dulcin, glucin, neohesperidin dihydrochalcone, neotame, p-4000, saccharin, L-rhamnose, l-glucose, l-mannose, l-fucose.

It is yet still another aspect of one advantageous form of the present formulation that the components be of bioactive phenolic nature. Examples of this include but are not limited to phenols, flavonals, flavonoids, bioflavonoids, anthoxanthins, flavanones, flavanols, flavans, anthocyanidins. Examples include: apiole, carnosol, carvacrol, dillapiole, rosemarinol, quercetin, kaempferol, myricetin, dihydromyricetin, fisetin, rutin, isorhamnetin, hesperitin, hesperidin, naringin, naringenin, silybin, eriodictyol, acacetin, apigenin, chrysin, diosmetin, tangeritin, luteolin, chrysanthemum, catechin, gallocatechin, epicatechin, epigallocatechin, epigallocatechin gallate, theaflavin, thearubigins, proanthocyanodins, pelagonidin, peonidin, cyanidin, delphinidin, malvidin, petunidin, daidzein, genistein, glycitien, isoflavanes, isoflavandiols, isoflavenes, pterocarpans, aurones, chalconoids, reseveratrol, pterostilbene, piceatannol, pinosylvan, ellagictannin, punicalagin, castalagin, vescalagin, castalin, casuarictin, grandinin, punicalins, roburin, tellimagrandin, terflavin, gallotannin, proanthocyanidin, polyflavonoid, pyrocatecolic, flavolan, salicylic acid, vanillin, gallic acid, elagic acid, tannic acid, caffeic acid, chlorogenic acid, ferulic acid, coumarin, tyrosol, hydroxytyrosol, oleocanthol, oleuropein, capsaicin, gingerol, maltodextrin, dextrose, sterubin, azalein, galangin, gossypetin, hyperoside, icariin, isoquercetin, isorhamnetin, kaempferide, kaempferitrin, morin, myricitrin, quercitrin, pachypodol, rhamnazin, robinin, robinose, spiraeoside, spirenoside, troxerutin, wogonin, sterubin, poncirin, eupafolin, apigenin.

It is still a further aspect of one advantageous form of the present formulation to contain a starch such as, but not limited, to a polymeric carbohydrate or polysaccharide. Such examples are but not limited to glucopyranose, amylose, amylopectin, glycogen, arabinoxylan, cellulose, chitin, pectin, acidic polysaccharides.

In yet another advantageous form of the present formulation, the phenol addictive acts to control the negative effects of hyperglycemic by the following methods: inhibition of pro-inflammatory tnf-α, inhibition of alpha amylase, inhibition of glucosidase, inhibition of glut 2 transport into adipose tissue, stimulation of glut 4 transport into muscle tissue, stimulation of mitochondria genesis in muscle tissue.

In still another aspect of one advantageous form of the present formulation, the formulation contains a suitable start for inhibiting the glycemic impact of sugars by the following methods: direct inhibition of the enzyme alpha amylase, direct inhibition of the enzyme glucosidase, inhibition and buffering of stomach acids.

In yet another aspect of a preferred formulation, the formulation prevents the cavity formation of the mouth by preventing or reducing enamel degrading acids from the byproduct of amylase activity. The phenolic ingredient of the improved sugar formulation is that sufficient enough to inhibit amylase activity in the oral cavity.

Further, in still another aspect of an advantageous form of the present formulation, the formulation results in weightloss or prevents weight gain though direct interaction of reducing fat accumulation by inhibition of the glut 2 pathway of adipose tissues and direct stimulation of increased energy/carbohydrate consumption by activation of the glut 4 pathway.

### EXEMPLAR PREFERRED FORMULATIONS EXAMPLES

Some preferred formulations include a combination of three components in effective amounts to mitigate or rectify hyperglycemic effects of high glycemic loads. This formulation may be accomplished in three different but functional aspects. The three distinct components are a sweetening agent, a polyphenol, and a starch. The formulation can exist as a combination of the three ingredients combined in a manner sufficient for addition into foodstuff such as a beverage, batter, or medicinal formulation. The formulation, in various advantageous form, can be re-crystalized into a formed compound using the sugar as the carrying agent for the remaining two compounds. Further, various advantageous forms of the present formulation be combined into a liquid aspect in which the compounds are carried by a liquid sweetener such as corn syrup.

Advantageous forms of the formulation include, but are not limited to the following two examples:

i) a sweetening agent, gallic acid as the polyphenol and arabinoxylan as the starch; and

ii) a sweetening agent, hesperitin or hesperidin as the polyphenol and inulin as the starch.

### EXAMPLES OF SOME PREFERRED FORMULATIONS

### Example 1

### Dry Formulation:

70%-95% sucrose (sweetener)

10%-30% gallic acid (polyphenol)

10%-30% aribinoxylan (starch)

### Example 2

### Crystalized Formulation:

Sucrose or other suitable sweetener is heated to sufficient temperature to liquefy. The second then third ingredients are then added to the heated and liquid phase of the sucrose. The mixture is allowed to dry and then finally granulized.

70%-95% sucrose (sweetener)

10%-30% gallic acid (polyphenol)

10%-30% aribinoxylan (starch)

### Example 3

### Liquid Base Formulation:

70%-95% corn syrup (liquid sweetener base)

10%-30% gallic acid (polyphenol)

10%-30% aribinoxylan (starch)

### Example 4

### Dry Formulation:

70%-95% sucrose (sweetener)

10%-30% hesperitin or hesperidin (polyphenol)

10%-30% inulin (starch)

### Example 5

### Crystalized Formulation:

Sucrose or other suitable sweetener is heated to sufficient temperature to liquefy. The second then third ingredients are then added to the heated and liquid phase of the sucrose. The mixture is allowed to dry and then finally granulized.

70%-95% sucrose (sweetener)

10%-30% hesperitin or hesperidin (polyphenol)

10%-30% inulin (starch)

### Example 6

### Liquid Base Formulation:

70%-95% Corn Syrup (liquid sweetener base)

10%-30% hesperitin or hesperidin (polyphenol)

10%-30% inulin (starch)

### EXEMPLARY METHOD FOR MANUFACTURING AND FURTHER FORMULATIONS ARE PROVIDED AS FOLLOWS

This formulation is for an improved sugar quantity of 1 kilogram or 1 liter with a combinational ratio of 80% sweetener, 10% Phenol and 10% Starch.

### Example Dry Formulation

800 g sucrose

100 g gallic acid

100 g aribinoxylan

Ingredients are mixed together to create a dry, combined formula.

### Example of Crystalized Formula

800 g of sucrose is heated to sufficient temperature to liquefy (215° F.-320° F. or 101° C.-160° C.).

100 g of gallic and 100 g of arabinoxylan is added and mixed into the liquid slurry.

The heated slurry is allowed to cool and then re-crystalize to form conventional sugar crystals with gallic acid (phenol) and arabinoxylan (starch) incorporated inside.

### Example of Liquid Formula

800 ml of corn syrup is heated above room temperature (110° F.-320° F. or 30° C.-160° C.) 100 g of gallic acid and 100 g arabinoxylan are added to the liquefied slurry and mixed or stirred. The slurry is then allowed to cool to room temperature.

This formulation is for an improved sugar quantity of 1 kilogram or 1 liter with a combinational ratio of 70% corn syrup (sweetener), 10% gallic acid (phenol) and 20% aribinoxylan (starch).

### Example Dry Formulation

700 g sucrose

100 g myricetin

200 g glucomannan

Ingredients are mixed together to create a dry, combined formula.

### Example of Crystalized Formula

800 g of sucrose is heated to sufficient temperature to liquefy (215° F.-320° F. or 101° C.-160° C.).

100 g of myricetin and 200 g of glucomannan is added and mixed into the liquid slurry. The heated slurry is allowed to cool and then re-crystalize to form conventional sugar crystals with myricetin (phenol) and glucomannan (starch) incorporated inside.

### Example of Liquid Formula

800 ml of corn syrup is heated above room temperature (110° F.-320° F. or 30° C.-160° C.) 100 g of myricetin and 200 g glucomannan are added to the liquefied slurry and mixed or stirred. The slurry is then allowed to cool to room temperature.

This formulation is for an improved sugar quantity of 1 kilogram or 1 liter with a combinational ratio of 90% corn syrup (sweetener), 5% myricetin (phenol) and 5% glucomannan (starch).

### Example Dry Formulation

900 g fructose

50 g hesperitin or hesperidin

50 g pectin

Ingredients are mixed together to create a dry, combined formula.

### Example of Crystalized Formula

900 g of fructose is heated to sufficient temperature to liquefy (215° F.-320° F. or 101° -160° C.).

50 g of hesperitin or hesperidin and 50 g of pectin is added and mixed into the liquid slurry.

The heated slurry is allowed to cool and then re-crystalize to form conventional sugar crystals with hesperitin or hesperidin (phenol) and pectin (starch) incorporated inside.

### Example of Liquid Formula:

900 ml of corn syrup is heated above room temperature (110° F.-320° F. or 30° C.-160° C.) 50 g of hesperitin or hesperidin and 50 g pectin are added to the liquefied slurry and mixed or stirred. The slurry is then allowed to cool to room temperature.

### Example Dry Formulation

800 g sucrose

100 g gallic acid

100 g aribinoxylan

Ingredients are mixed together to create a dry, combined formula.

### Example of Crystalized Formula

800 g of sucrose is heated to sufficient temperature to liquefy (215° F.-320° F. or 101° C.-160 ° C.).

100 g of gallic and 100 g of arabinoxylan is added and mixed into the liquid slurry. The heated slurry is allowed to cool and then re-crystalize to form conventional sugar crystals with gallic acid (phenol) and arabinoxylan (starch) incorporated inside.

### Example of Liquid Formula

800 ml of corn syrup is heated above room temperature (110° F.-320° F. or 30° C.-160° C.) 100 g of gallic acid and 100 g arabinoxylan are added to the liquefied slurry and mixed or stirred. The slurry is then allowed to cool to room temperature.

This formulation is for an improved sugar quantity of 1 kilogram or 1 liter with a combinational ratio of 70% corn syrup (sweetener), 10% gallic acid (phenol) and 20% arabinoxylan (starch).

### Example Dry Formulation

800 g sucrose

100 g hesperitin or hesperidin

100 g inulin

Ingredients are mixed together to create a dry, combined formula.

### Example of Crystalized Formula

800 g of sucrose is heated to sufficient temperature to liquefy (215° F.-320° F. or 101° C.-160 ° C.).

100 g of hesperitin or hesperidin and 100 g of inulin is added and mixed into the liquid slurry.

The heated slurry is allowed to cool and then re-crystalize to form conventional sugar crystals with hesperitin or hesperidin and inulin incorporated inside.

### Example of Liquid Formula

800 ml of corn syrup is heated above room temperature (110° F.-320° F. or 30° C.-160° C.) 100 g of hesperitin or hesperidin and 100 g inulin are added to the liquefied slurry and mixed or stirred. The slurry is then allowed to cool to room temperature.

This formulation is for an improved sugar quantity of 1 kilogram or 1 liter with a combinational ratio of 70% corn syrup (sweetener), 10% hesperitin or hesperidin (phenol) and 20% inulin (starch).

In addition to these examples it will be apparent to one of ordinary skill in the art that modifications can be made which are consistent with this disclosure. For example, a formulation can include more than one sweetener, more than one polyphenol and more than one starch, such that a combination of sweeteners, polyphenols and/or starches may be present in a single formulation.

What is claimed is:

1. A formulation comprising a sweetening agent, a polyphenol and a starch.

2. The formulation of claim 1 wherein the formulation mitigates the effects of hyperglycemic when administered to an individual in need thereof.

3. The formulation of claim 2 wherein the hypoglycemia condition is selected from the group consisting of metabolic syndrome, obesity, diabetes and neurological disease.

4. The formulation of claim 1 further comprising food stuff including beverages, breads, candies, confections, coatings, medical foods and medicinal formulations.

5. The formulation of claim 1 wherein the composition has been heated and the sweetening agent liquefied so that the polyphenol and starch are embedded in a re-crystalized form of the sweetening agent.

6. The formulation of claim 5 wherein the formulation has been incorporated into table sugar, beverages, breads, candies, confections, coatings, medical foods and medicinal formulations.

7. The formulation of claim 1 wherein the formulation is disbursed and carried in a liquid phase of the sweetening agent such as corn syrup.

8. The formulation of claim 7 wherein the formulation is incorporated into beverages, breads, candies, confections, coatings, medical foods and medicinal formulations.

9. The formulation of claim 1 wherein the sweetening agent is a carbohydrate base both a natural and semi synthetic nature including monosaccharides, disaccharides, oligosaccharides and artificial sweeteners, including fructose, galactose, sucrose, deoxyribose, diose, triose, tetrose, pentose, hexose, pentose, aldose, furanose, pyranose, glucofuranose, glucopyranose, sucrose, lactulose, lactose, maltose, cellobiose, chitobiose, kojibiose, nigerlose, isomaltose, sophorose, laminiaribiose, gentiobiose, turanose, maltulose, palatinose, mannobiose, melibiose, melibiulose, runtinose, xylobiose, fructo-oligosaccharide, galacto-oligosaccharide, manno-oligosaccharide, aspartame, cyclamate, saccharin, stevia, sucralose, acesulfame potassium, lead acetate, mogrosides, brazzein, curculin, erythritol, glycyrrhizin, glycerol, inulin, isomalt, lactitol, mabinlin, maltitol, miraculin, monatin, monellin, osladin, pentadin, sorbitol, tagetose, thaumatin, xylitol, advantame, alitame, dulcin, glucin, neohesperidin dihydrochalcone, neotame, p-4000, saccharin, l-rhamnose, l-glucose, l-mannose, l-fucose.

10. The formulation of claim 1 wherein at least one of the sweetening agent, polyphenol and starch have a bioactive phenolic nature which include but is not limited to phenols, flavonals, flavonoids, bioflavonoids, anthoxanthins, flavanones, flavanols, flavans, anthocyanidins. Examples include; apiole, carnosol, carvacrol, dillapiole, rosemarinol, quercetin, kaempferol, myricetin, dihydromyricetin, fisetin, rutin, isorhamnetin, hesperitin, hesperidin, naringin, naringenin, silybin, eriodictyol, acacetin, apigenin, chrysin, diosmetin, tangeritin, luteolin, chrysanthemum, catechin, gallocatechin, epicatechin, epigallocatechin, epigallocatechin gallate, theaflavin, thearubigins, proanthocyanodins, pelagonidin, peonidin, cyanidin, delphinidin, malvidin, petunidin, daidzein, genistein, glycitien, isoflavanes, isoflavandiols, isoflavenes, pterocarpans, aurones, chalconoids, reseveratrol, pterostilbene, piceatannol, pinosylvan, ellagictannin, punicalagin, castalagin, vescalagin, castalin, casuarictin, grandinin, punicalins, roburin, tellimagrandin, terflavin, gallotannin, proanthocyanidin, polyflavonoid, pyrocatecolic, flavolan, salicylic acid, vanillin, gallic acid, elagic acid, tannic acid, caffeic acid, chlorogenic acid, ferulic acid, coumarin, tyrosol, hydroxytyrosol, oleocanthol, oleuropein, capsaicin, gingerol, maltodextrin, dextrose, sterubin, azalein, galangin, gossypetin, hyperoside, icariin, isoquercetin, isorhamnetin, kaempferide, kaempferitrin, morin, myricitrin, quercitrin, pachypodol, rhamnazin, robinin, robinose, spiraeoside, spirenoside, troxerutin, wogonin, sterubin, poncirin, eupafolin, apigenin.

11. The formulation of claim 1 wherein the starch is selected from the group consisting of polymeric carbohydrate or polysaccharide, including glucopyranose, amylose, amylopectin, glycogen, arabinoxylan, cellulose, chitin, pectin, acidic polysaccharides.

12. This formulation of claim 1 wherein the polyphenol is adapted, when the formulation administered to an individual, to control negative effects of hyperglycemic by one or more of the following methods: inhibition of pro-inflammatory tnf-α, inhibition of alpha amylase, inhibition of glucosidase, inhibition of glut 2 transport into adipose tissue, stimulation of glut 4 transport into muscle tissue and/or stimulation of mitochondria genesis in muscle tissue.

13. The formulation of claim 1 wherein the formulation when administered to an individual inhibits glycemic impact of sugars by one or more of the following methods: direct inhibition of the enzyme alpha amylase, direct inhibition of the enzyme glucosidase, inhibition and buffering of stomach acids.

14. The formulation of claim 1 wherein the formulation when administered to an individual in need thereof prevents cavity formation of the mouth by preventing or reducing enamel degrading acids from the byproduct of amylase activity.

15. The formulation of claim 1 wherein the polyphenol is formulated to inhibit emulase activity in the oral cavity when the formulation is administered to an individual in need thereof.

16. The formulation of claim l wherein when administered to a patient in need thereof results in improved sugar formulation will cause weightloss or prevent weight gain though direct interaction of reducing fat accumulation by inhibition of the glut 2 pathway of adipose tissues and direct stimulation of increased energy/carbohydrate consumption by activation of the glut 4 pathway.

17. The formulation of claim 1, wherein the polyphenol is hesperitin or hesperidin and the starch is inulin.

18. The formulation of claim 1, wherein the polyphenol is gallic acid and the starch is arabinoxylan.
